(19) United States
(12) Patent Application Publication
(10) Pub. No.: US 2021/0118545 A1
(43) Pub. Date: Apr. 22, 2021

(54) SYSTEM AND METHOD FOR RECOMMENDING FOOD ITEMS BASED ON A SET OF INSTRUCTIONS

(71) Applicants: Suchitra SATHYANARAYANA, Sunnyvale, CA (US); Ravi Kumar SATZODA, Sunnyvale, CA (US)

(72) Inventors: Suchitra SATHYANARAYANA, Sunnyvale, CA (US); Ravi Kumar SATZODA, Sunnyvale, CA (US)

(21) Appl. No.: 17/072,874

(22) Filed: Oct. 16, 2020

Related U.S. Application Data
(60) Provisional application No. 62/923,257, filed on Oct. 18, 2019.

(51) Int. Cl. G16H 20/60; G16H 50/30; G06N 5/04; G06F 40/232

(52) U.S. Cl. CPC G16H 20/60; G06F 40/232; G06N 5/04; G16H 50/30

(57) ABSTRACT

A system and method is provided for recommending food items based on a set of instructions. A first set of instructions are executed to receive a first set of input parameters associated with plurality of attributes of the entity. Further, a second set of input parameters are received from a second entity and are associated with the first set of input parameters of the entity. The received first set of input parameters and the received second set of input parameters are analyzed to determine at least one of a health label for the entity. Then, a health score is assigned for at least one of the health label for the entity. The health score is assigned based on a food item to be recommended. Upon, the assigned health score lying within a predefined threshold, the food item is recommended to the entity.

### RELATED APPLICATION

This application claims priority from U.S. Provisional Application Ser. No. 62/923,257, entitled “SYSTEM AND METHOD FOR RECOMMENDING FOOD ITEMS BASED ON A SET OF INSTRUCTIONS,” filed on Oct. 18, 2019, the contents of which are hereby incorporated herein in its entirety by this reference.

### TECHNICAL FIELD

The present disclosure relates generally to recommending food items to an entity. In particular, it relates to providing recommendations related to food items based on multiple input parameters received from a patient and a health expert.

### BACKGROUND OF THE INVENTION

In a fast pace of life that is being witnessed today, most people find it difficult to maintain or improve their health. Even when the patient has a desire to improve his/her health, he/she may not be sensitive of what alterations are required in lifestyle, what kind of exercise regime should be followed and what diet plan should be adopted to make desired improvement in health conditions. Further, even when being informed about preventive measures regarding a better diet and a healthy lifestyle, many patients have trouble following through with lifestyle resolutions such as to eat healthy and exercise. Even though when the patient knows what general changes are needed to be made in lifestyle to make the desired improvement in health, he/she may not know how to bring about the necessary changes. There are many different tools and services available in the market today to cater to recommending changes to dietary lifestyle.

Prevalent approaches use recipe recommendation approach by recommending recipes for nutritious meals recommended to users in order to achieve a healthy diet. There are perhaps millions or even billions of different recipes available in various publications such as recipe books, magazines, health books, and online recipe databases. One common problem faced by users is selecting an appropriate recipe from among the overwhelming number of choices available.

In another scenario, dietary recommendations provided to users are generally focused to achieve weight loss. Such recommendation systems consider general calorific requirement of the user and recommend meals to enable the user to achieve weight loss. Another, recipe recommendation program is based on general inputs regarding the available ingredients and preferences of the users. They focus on enabling users to prepare meals by using the available ingredients.

However, such recipe recommendation system fail to understand a holistic requirement of the users based on their health history, current health status, preferences and so on. Therefore, there is a requirement for an efficient recipe recommendation which is based on the consumer's preferences and healthy and unhealthy habits.

### SUMMARY

The present disclosure relates generally to recommending food items to an entity. In particular, it relates to providing recommendations related to food items based on multiple input parameters received from a patient and a health expert.

According to an aspect of the present disclosure is provided a method for recommending a food item to an entity, said method comprising: receiving, at a processor of a remote computing device executing a first set of instructions, a first set of input parameters ri associated with the entity, the first set of input parameters being indicative of one or more attributes of the entity and representative of one or more continuous variables; receiving, at the processor executing the first set of instructions, a second set of input parameters from a second entity, the second set of input parameters being indicative of one or more health categories cj, where the one or more health categories are associated with the first set of input parameters of the entity, the health categories being representative of one or more categorical variables; analyzing, at the processor executing the first set of instructions, the received first set of input parameters and the received second set of input parameters to determine at least one of a health label for the entity; assigning, at the processor executing a second set of instructions, a health score for at least one of the health label for the entity, the health score being assigned based on a food item to be recommended; and upon the assigned health score being within a predefined threshold, recommending, at the processor, the food item to the entity.

According to an embodiment, the represented one or more continuous variables are real value numbers and are provided as input to the first set of instructions, and wherein the first set of input parameters representative of the one or more continuous variables are received using questionnaire data provided by the entity.

According to an embodiment, the method further comprises: extracting, at the processor, a sequence of words from the food item to be recommend to the entity; determining, at the processor, a plurality of similar words based on the extracted sequence of words using a third set of instructions, the determined plurality of similar words being indicative of the food item to be recommend to the entity, and wherein executing the third set of instructions to map the food item to be recommend to at least one of the health label ; and receiving, at the processor, the health score for at least one of the health label, wherein upon the received health score being within the predefined threshold, recommending the food item to the entity.

According to an embodiment, upon the determined plurality of similar words being indicative of the food item to be recommend to the entity and having being mapped to at least one of the health label and having the received health score lying within the predefined threshold, storing, at the processor, the determined plurality of similar words in a dataset, wherein each of the determined plurality of similar words are indicative of the food item to be recommend to the entity and is mapped to at least one of the health label.

According to an embodiment, receiving, at the processor, a set of entity preferences, the health label for the entity, and at least one of an ingredient for a recipe, where the recipe is indicative of a collection of multiple food items; executing, at the processor using the third set of instructions, the received set of entity preferences, the health label, and at least one of the ingredient for the recipe to determine a second health score, and upon the determined second score being within the predefined threshold, recommending, at the processor, the recipe for consumption to the entity.

According to an embodiment, the determined plurality of similar words comprises at least one of a misspelling, synonym, abbreviation, metonym, synecdoche, metalepsis, kenning, or acronym associated with the extracted sequence of words.

According to an embodiment, the method further comprises determining, at the processor, a difference level between the extracted sequence of words and the determined plurality of similar words, and if the difference level between the extracted sequence of words and the at least one of determined plurality of similar words is below a threshold value considering the at least one of determined plurality of similar words as a closest match to the extracted sequence of words.

According to an embodiment, the first set of instructions comprises any of machine learning model, an XGBoost based decision tree model, and a random forest model.

According to an embodiment, the second set of instructions and the first set of instructions are optimized for execution using an L2 loss function, where the L2 loss function is represented as

$$L = \frac{1}{N} {\sum }_{\text{?}} ( Li ) {,}_{=} \text{?} \sum {\text{?}}_{2} ) , \\ \text{?} \text{indicates text missing or illegible when filed}$$

and where [symbol] is a ground truth output label, f is a machine learning model that maps an input x [symbol] to an output, the output being indicative of the health score to be assigned based on the food item to be recommended.

According to an embodiment, the output is a numerical value in a range of 1 to 3.

According to an embodiment, the output value of 1 is indicative of a low health score, 2 is indicative of a neutral health score, and 3 is indicative of a high health score.

According to an embodiment, the third set of instructions comprises any of a neural network language model, and natural language processing mechanism.

According to an embodiment, the one or more continuous variables and the one or more categorical variables are represented as an n-dimensional input vector x, where x={r₀, r₁, . . . , rᵢ, . . . , rₖ, cₖ₊₁, c₂, . . . , cⱼ. . . cₙ₋₁}, and where r_{i′}. R, and 0≤i≤k, and cⱼ [symbol] C, and k+1≤j≤n−1, and the variables are indicative of an association of the entity to at least one of the health label.

According to an embodiment, the health score is updated upon a change being determined in the received first set of input parameters and on receiving one or more instructions from the second entity.

According to an embodiment, the one or more attributes of the entity corresponds to any or a combination of behavioral, emotional and physical characteristics of the entity.

According to an aspect of the present disclosure is provided a system for recommending a food item to an entity, said system comprising: a processor of a remote computing device operatively coupled to a memory, the memory storing a first set of instructions and a second set of instructions executed by the processor to: receive a first set of input parameters ri associated with the entity, the first set of input parameters being indicative of one or more attributes of the entity and representative of one or more continuous variables; receive a second set of input parameters from a second entity, the second set of input parameters being indicative of one or more health categories cj, where the one or more health categories are associated with the first set of input parameters of the entity, the health categories being representative of one or more categorical variables; analyze the received first set of input parameters and the received second set of input parameters to determine at least one of a health label for the entity; assign a health score for at least one of the health label for the entity, the health score being assigned based on a food item to be recommended, the assignment being done on the execution of the second set of instructions; and upon the assigned health score being within a predefined threshold, recommend the food item to the entity.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

The accompanying drawings are included to provide a further understanding of the present disclosure and are incorporated in and constitute a part of this specification. The drawings illustrate exemplary embodiments of the present disclosure and, together with the description, serve to explain the principles of the present disclosure.

FIG. 1A illustrates exemplary network architecture in which or with which a food item recommendation system be implemented in accordance with an embodiment of the present disclosure.

FIG. 1B illustrates exemplary functional components of the food item recommendation system in accordance with an embodiment of the present disclosure.

FIG. 2A illustrates a high level architecture of a natural language processing, based on recipe score recommender in accordance with an embodiment of the present disclosure.

FIG. 2B illustrates determining word embeddings obtained from deep neural network in accordance with an embodiment of the present disclosure.

FIG. 3 illustrates a framework of the food item recommendation system, in accordance with an embodiment of the present disclosure.

FIG. 4 illustrates a detailed framework of the food item recommendation system, in accordance with an embodiment of the present disclosure.

FIG. 5 illustrates a deep learning-based recommendation engine in the food item recommendation system in accordance with an embodiment of the present disclosure.

FIG. 6 illustrates a 3D joint estimation technique for posture determination of a patient in accordance with an embodiment of the present disclosure.

FIG. 7 is a flow diagram illustrating a food item recommendation processing in accordance with an embodiment of the present invention.

FIG. 8 is an exemplary computer system in which or with which embodiments of the present invention may be utilized.

### DETAILED DESCRIPTION

The following is a detailed description of embodiments of the disclosure depicted in the accompanying drawings. The embodiments are in such details as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

Embodiments of the present invention may be provided as a computer program product, which may include a machine-readable storage medium tangibly embodying thereon instructions, which may be used to program a computer (or other electronic devices) to perform a process. The term “machine-readable storage medium” or “computer-readable storage medium” includes, but is not limited to, fixed (hard) drives, magnetic tape, floppy diskettes, optical disks, compact disc read-only memories (CD-ROMs), and magneto-optical disks, semiconductor memories, such as ROMs, PROMs, random access memories (RAMs), programmable read-only memories (PROMs), erasable PROMs (EPROMs), electrically erasable PROMs (EEPROMs), flash memory, magnetic or optical cards, or other type of media/machine-readable medium suitable for storing electronic instructions (e.g., computer programming code, such as software or firmware).

A machine-readable medium may include a non-transitory medium in which data may be stored and that does not include carrier waves and/or transitory electronic signals propagating 10 wirelessly or over wired connections. Examples of a non-transitory medium may include, but are not limited to, a magnetic disk or tape, optical storage media such as compact disk (CD) or digital versatile disk (DVD), flash memory, memory or memory devices. A computer-program product may include code and/or machine-executable instructions that may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, etc.

Furthermore, embodiments may be implemented by hardware, software, firmware, middleware, microcode, hardware description languages, or any combination thereof When implemented in software, firmware, middleware or microcode, the program code or code segments to perform the necessary tasks (e.g., a computer-program product) may be stored in a machine-readable medium. A processor(s) may perform the necessary tasks.

Systems depicted in some of the figures may be provided in various configurations. In some embodiments, the systems may be configured as a distributed system where one or more components of the system are distributed across one or more networks in a cloud computing system.

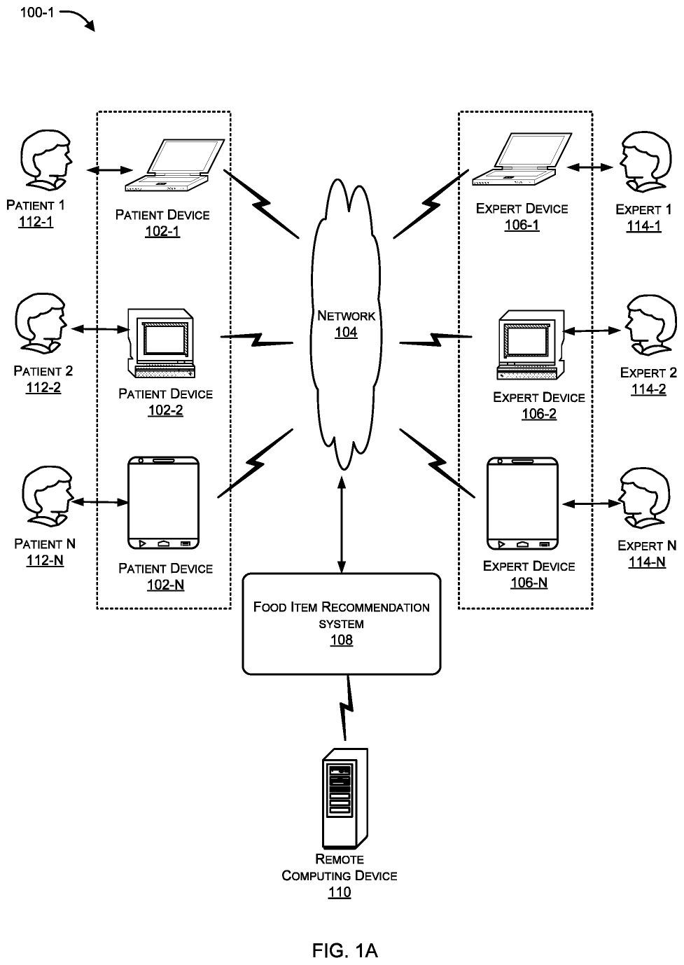

FIG. 1A illustrates exemplary network architecture 100-1 in which or with which a food item recommendation system be implemented in accordance with an embodiment of the present disclosure.

In context of the present exemplary architecture 100-1, a food item recommendation system 108 (also referred to as the system 108, hereinafter) is described. The system 108 can be implemented in any computing device and can be configured/operatively/communicably connected with a server 110. As illustrated, patients 112-1, 112-2, . . . , 112N (individually referred to as the patient 112 and collectively referred to as the patients 112, hereinafter) can interact with the system 108 using respective patient devices 102-1, 102-2, . . . , 102-N (individually referred to as the patient device 102 and collectively referred to as the patient devices 102, hereinafter), which can be communicatively coupled with the system 108 through a network 104. Further, the system 108 can be communicatively coupled with one or more expert devices 106-1, 106-2, . . . , 106-N of a caregiver team (individually referred to as the expert device 106 and collectively referred to as the experts devices 106, hereinafter) through the network 104, which can enable the experts 114-1, 114-2, . . . , 114N (individually referred to as the expert 114 and collectively referred to as the experts 114, hereinafter) to interact with the system 108. The patient devices 102 and the expert devices 106 can include a variety of computing systems, including but not limited to, a laptop computer, a desktop computer, a notebook, a workstation, a portable computer, a personal digital assistant, a handheld device and a mobile device. In an embodiment, the system 108 can be implemented using any or a combination of hardware components and software components such as a cloud, a server, a computing system, a computing device, a network device and the like.

Further, the system 108 can interact with the patient device 102 and the expert device 106 through a website or an application residing at the patient devices 102 and the expert devices 106. Further, front end part of the application can be implemented using hypertext markup language (HTML), Java scripting and suitable languages to enable the experts 114 to interact with the patients 112 using a video conferencing software, to enable the experts 114 to input their food item recommendation plans during consultations and to enable visualization of patient's health record. Further, the front end part of the application can be implemented to enable the patient device 102 receive input from the patient 112 in relation to patient's current lifestyle and present health conditions in form of a questionnaire presented on a patient's device 102. A backend part of the application can be implemented as a database management system and data analytics can be performed on the patient's data. The backend application can be implemented using Node.JS, AngularJS, Amazon Web Services (AWS), Machine Learning (ML), and infrastructures such as Amazon SageMaker, custom ML implementations, MS Azure® or Google Cloud™ AI. The system 108 can provide an administration panel that combines the frontend and backend part of the application and enables providing personalized food item recommendation to the patients 112 through the experts 114. Further, the front end can be implemented using technologies like Node.JS, AngularJS, Amazon Web Services (AWS), etc. Further, the system 108 can be accessed by the website or the application that can be configured with any operating system, including but not limited to, Android™, iOS™, and the like.

In an embodiment, the system 108 facilitates to determine patient's health status information based on the patient's lifestyle, dietary, exercise regime, and physiological parameters based on a machine-learning model. Further, analysis is performed on the determined patient's health status information to extract lifestyle trends of the patient. Based on the determined patient's health status and the extracted lifestyle trends of the patient, a health care plan including food item recommendation by an expert 114 of a health care team is provided. The machine-learning model implemented by the system 108 calculates a health score depicting patient's health status. The health score is used along with the extracted lifestyle trends of the patient to recommend appropriate dietary routine that specifically includes recommending the food item to be consumed by the entity. The system 108 may provide the personalized dietary recommendations through various modules implementing but not limited to machine learning models and natural language processing based models, that have been discussed in detail in the specification hereafter.

In an embodiment, a first set of input parameters rᵢ associated with the entity 112-1 (e.g., the patient) are received. The first set of input parameters are indicative of one or more attributes of the entity 112-1 and are representative of one or more continuous variables. The one or more attributes of the entity may correspond to any or a combination of behavioral, emotional and physical characteristics of the entity 112-1. The represented one or more continuous variables may be real value numbers and may be provided as input to the first set of instructions, and wherein the first set of input parameters are representative of the one or more continuous variables and are received using questionnaire data provided by the entity 112-1. In an embodiment, the first set of instructions may include any of machine learning model, an XGBoost based decision tree model, and a random forest model.

Further, a second set of input parameters are received from a second entity 114-1 (e.g., health care expert) associated with the entity 112-1, the second set of input parameters being indicative of one or more health categories cj, where the one or more health categories are associated with the first set of input parameters of the entity 112-1, the health categories being representative of one or more categorical variables. The received first set of input parameters and the received second set of input parameters are analyzed to determine at least one of a health label for the entity 112-1. In an embodiment, the one or more continuous variables and the one or more categorical variables may be represented as an n-dimensional input vector x, where x={r₀, r₁, . . . , rᵢ, . . . , rₖ, cₖ₊₁, c₂, . . . , cⱼ, . . . , cₙ₋₁}, and where rᵢ. R, and 0≤i≤k, and cⱼ [symbol] C, and k+1≤j≤n−1 for recommending the food item to the entity.

Subsequently, a second set of instructions are analyzed to assign a health score for at least one of the health label for the entity 112-1. The health score is assigned based on a food item to be recommended. Upon the assigned health score being within a predefined threshold, the food item is recommended to the entity 112-1. In an embodiment, the execution of the second set of instructions and the first set of instructions is optimized using an L2 loss function. The L2 loss function may be represented as

$$L = \text{?} \sum \text{?} , = \frac{1}{N} {\sum }_{\text{?}} ( {( f ( {x}_{i} ) - {\hat{y}}_{i} )}_{2} ) , \\ \text{?} \text{indicates text missing or illegible when filed}$$

and where is a ground truth output label, f(x [symbol] ) is a machine learning model that maps an input to an output, the output may indicate the health score to be assigned based on the food item to be recommended. The output may be a numerical value in a range of 1 to 3, where the output value of 1 may indicate a low health score, 2 may indicate a neutral health score, and 3 may indicate a high health score. In an embodiment, the execution of the first set of instructions and the second set of instructions may be optimized using any of a L2 loss function, a L1 loss function, a huber loss function, and a classification problem with a log loss function and a softmax loss function. In yet another embodiment, the health score may be updated when a change is determined in the received first set of input parameters and when one or more instructions are received from the second entity.

In an embodiment, the system 108 facilitates to extract a sequence of words from the food item to be recommend to the entity 112-1. Next a plurality of similar words based on the extracted sequence of words is determined using a third set of instructions. The determined plurality of similar words may be indicative of the food item to be recommended to the entity. The third set of instructions may be executed based on the received one or more continuous variables and the one or more continuous categorical variables. In an embodiment, the third set of instructions may include any of a neural network language model, and natural language processing mechanism.

Further, the health score for each of the plurality of the similar words may be received. The determined plurality of similar words may include at least one of a misspelling, synonym, abbreviation, metonym, synecdoche, metalepsis, kenning, or acronym associated with the extracted sequence of words. Upon the received health score for at least one of the word of the plurality of the similar words being within the predefined threshold, the food item associated to the at least one of the word to the entity may be recommended. In an embodiment, the system m108 facilitates to include determining a difference level between the extracted sequence of words and the determined plurality of similar words, and if the difference level between the extracted sequence of words and the at least one of determined plurality of similar words is below a threshold value, the at least one of determined plurality of similar words may be determined as a closest match to the extracted sequence of words.

Those skilled in the art would appreciate that network 104 can be wireless network, wired network or a combination thereof that can be implemented as one of the different types of networks, such as Intranet, Local Area Network (LAN), Wide Area Network (WAN), Internet, and the like. Further, network 104 can either be a dedicated network or a shared network. The shared network represents an association of the different types of networks that use a variety of protocols, for example, Hypertext Transfer Protocol (HTTP), Transmission Control Protocol/Internet Protocol (TCP/IP), Wireless Application Protocol (WAP), and the like.

FIG. 1B illustrates exemplary functional components 100-2 of the food item recommendation system in accordance with an embodiment of the present disclosure.

In an aspect, the system 108 may comprise one or more processor(s) 112. The one or more processor(s) 112 may be implemented as one or more microprocessors, microcomputers, microcontrollers, digital signal processors, central processing units, logic circuitries, and/or any devices that manipulate data based on operational instructions. Among other capabilities, the one or more processor(s) 112 are configured to fetch and execute computer-readable instructions stored in a memory 116 of the system 108. The memory 116 may store one or more computer-readable instructions or routines, which may be fetched and executed to create or share the data units over a network service. The memory 116 may comprise any non-transitory storage device including, for example, volatile memory such as RAM, or non-volatile memory such as EPROM, flash memory, and the like.

The system 108 may also comprise an interface(s) 114. The interface(s) 114 may comprise a variety of interfaces, for example, interfaces for data input and output devices, referred to as I/O devices, storage devices, and the like. The interface(s) 114 may facilitate communication of the system 108 with various devices coupled to the system 108. The interface(s) 114 may also provide a communication pathway for one or more components of the system 108. Examples of such components include, but are not limited to, processing engine(s) 118 and database 120.

In some embodiments, the recommendations further may be delivered to the patient 112 in written form, spoken form, visual form, tactile form, and the like. The recommendations may be delivered to said subject in a variety of ways, e.g., by screen display, printed output, text message, email, an audible reminder signal, buzzer, instant messaging, social media, message boards/blogs, or other suitable private or user-authorized public form of communication.

The processing engine(s) 118 may be implemented as a combination of hardware and programming (for example, programmable instructions) to implement one or more functionalities of the processing engine(s) 118. In examples described herein, such combinations of hardware and programming may be implemented in several different ways. For example, the programming for the processing engine(s) 118 may be processor executable instructions stored on a non-transitory machine-readable storage medium and the hardware for the processing engine(s) 118 may comprise a processing resource (for example, one or more processors), to execute such instructions. In the present examples, the machine-readable storage medium may store instructions that, when executed by the processing resource, implement the processing engine(s) 118. In such examples, the system 108 may comprise the machine-readable storage medium storing the instructions and the processing resource to execute the instructions, or the machine-readable storage medium may be separate but accessible to the system 108 and the processing resource. In other examples, the processing engine(s) 118 may be implemented by electronic circuitry.

The database 120 may comprise data that is either stored or generated as a result of functionalities implemented by any of the components of the processing engine(s) 118. In an embodiment, the database 120 may include machine-learning based training database. In an embodiment, the training database may include a predefined mapping defining a relationship between various input parameters and output parameters based on various statistical methods. In an embodiment, the training database may include machine-learning algorithms to learn mappings between input parameters related to patient such as but not limited to physiological parameters, patient's health record, patient's lifestyle pattern, etc. and expert provided input.

In an embodiment, the training database may include a dataset which may include data collections that are not subject specific, i.e., data collections based on population-wide observations, local, regional or super-regional observations, and the like. Exemplary datasets include environmental information, drug interaction information, geographic data, climate data, meteorological data, retail data, pharmacy data, insurance data, market data, encyclopedias, scientific- and medical-related periodicals and journals, business information, research studies data, scientifically-curated genetics-related information, nutritional data, exercise data, physician and hospital/clinic location information, physician billing information, physician re-imbursement information, restaurant and grocery store location information, and the like. In an embodiment, training database is routinely updated and/or supplemented based on machine learning methods.

In an embodiment, in order for the machine learning models described in the current disclosure to recommend recipes based on Ayurvedic principles, a dataset is created that maps a food recipe to each of the five parameters—kapha, vata, pitta, aama and agni. In an exemplary embodiment, the dataset may be created by an ayurvedic expert who have gone through each recipe and have ranked each recipe for the 5 output parameters with a rating from 1 to 3. If a recipe is rated as 1 for a parameter, say kapha, it may mean the recipe reduces kapha, similarly 2 means neutral or no impact, and 3 means increases kapha.

To create the dataset of recipes, the ayurvedic experts may have gone through the ingredients of each recipe, quantities of the ingredients and steps in the recipe. Based on the above parameters, each recipe is then scored across the 5 different parameters. In an embodiment, the dataset may be extended to cater to other parameters (beyond the 5 listed above) that Ayurvedic experts are interested in. In an embodiment, the database 120 may be queried by the processing engine(s) to compute various outputs.

In an exemplary embodiment, the processing engine(s) 118 may comprise a first input parameters receiving engine 122, a second input parameters receiving engine 124, an input parameters analyzing engine 126, a health score assigning engine 128, a food item recommendation engine 130, and other engine(s) 132.

It would be appreciated that engines being described are only exemplary engines and any other engine or sub-engine may be included as part of the system 108 or the processing engine 118. These engines too may be merged or divided into super-engines or sub-engines as may be configured.

### First Input Parameters Receiving Engine 122

In an embodiment, a first set of input parameters rᵢ associated with the entity 112-1 (e.g., the patient) are received. The first set of input parameters are indicative of one or more attributes of the entity 112-1 and are representative of one or more continuous variables. The one or more attributes of the entity may correspond to any or a combination of behavioral, emotional and physical characteristics of the entity 112-1. The represented one or more continuous variables may be real value numbers and may be provided as input to the first set of instructions, and wherein the first set of input parameters are representative of the one or more continuous variables and are received using questionnaire data provided by the entity 112-1. In an embodiment, the first set of instructions may include any of machine learning model, an XGBoost based decision tree model, and a random forest model.

In an embodiment, health of the patient 112 can be monitored via a variety of monitoring devices. The monitoring devices can measure the patient's physiological parameters such as heart rate, blood oxygen saturation levels, respiratory rate, weight glucose level, blood pressure, weight, etc. The monitoring devices can be such as but not limited to a smart watch, wristband, smart phone, meditation band, smart clothing, or other devices including sensors capable of capturing the patient's activity data and vital data. The monitoring devices may include but not limited to a gyroscope, accelerometer, magnetometer, infrared sensor, camera, microphone, gas sensor, photo-detector, etc.

In an embodiment, the monitoring devices can be equipped with operating systems like Android™, iOS™, windows® and linux™ OS, or hybrid frameworks like React Native that enables efficient integration of the mobile and/or the wearable devices. A mobile application can be provided that is used on the mobile device such as the smart phone for enabling interaction of the patients with the experts of the caregiver team.

In an example, the monitoring device may record physiological data associated with the patient. The physiological data may include, for example, the patient's heart rate, blood pressure, etc. The determined physiological data along with a health care plan recommended by an expert of a health care team may be used as for computing the patient's health score. Status of the patient's physiological data may be determined (e.g., calculated) based on a difference between at least one characteristic of the physiological data and at least one characteristic of historic physiological data associated with the patient

In an embodiment, lifestyle pattern of the patient can be tracked and used along with the determined physiological parameters and health care plan recommended by an expert of a health care team to determine the health score of the patient. The lifestyle pattern of the patient can be determined by recognizing the patient's activity, which can be performed by determining parameters such as but not limited to detecting eating/drinking patterns, sleep detection, exercise detection, and detection of other activities such as smoking of the patient. In an embodiment, determination and collection of the patient's lifestyle pattern can be automated. Automation can be related to data collection, storage and processing, and helps achieve accuracy, is cost-efficient, and helps in maintaining a knowledge repository of the patient health related data. Further, in an embodiment, the lifestyle pattern of the patient can be determined using an automatic human activity recognition technique that captures data from the wearable and/or non-wearable monitoring devices. The human activity recognition technique can be used to build Human Activity Recognition (HAR) datasets. The lifestyle pattern can also include detecting and determining activities of interest such as related to diet, yoga poses, etc. using inertial measurement unit (IMU) sensors and camera.

Further, binge eating and junk food eating pattern of the patient can be determined by tracking dietary pattern and food item purchased by the patient. For example, the patient's frequent junk food purchases and consumption can be determined by auto tagging of the food to be consumed by the patient by using a camera. In addition, the patient's consumption of alcohol and/or other sweetened drinks can be determined by tagging of the drinks.

In an embodiment, sleep detection patterns of the patient can be determined by monitoring the patient's sleep by using techniques such as but not limited to an inertial measurement unit (IMU), heart rate data, respiratory rate and by capturing screen activity of the patient. Further, sleep data and sleep environment data of the patient can be used to detect and determine the patient's sleep quality.

In an embodiment, additional psychological patterns for the patient can be determined and can include, for example, emotion, mood, feeling, anxiety, stress, depression, and other psychological or mental states. The psychological patterns can be received as signals from the patients. For example, the patient can provide inputs related to the psychological patterns such as the patient being “angry”, “happy”, “sad”, “fearful” etc.

In an embodiment, symptoms of the patient such as related to pain, anxiety, happiness, fear can be determined to have co-relation with physiological information of the patient. Further, the symptoms can be fed into the system 108 to determine lifestyle patterns of the patient. Furthermore, the symptoms can be automatically inferred by the system 108 based on parameters such as related to exercise, diet, sleep and food intake of the patient 112. In an embodiment, exercise regime of the patient can be tracked by monitoring the patient's physical movement in context of running, walking and other physical activities, which can be measured by using the IMU technologies. Also, determination of whether the patient is practicing exercises such as yoga which are recommended and which kind of yoga asana can be performed.

In an embodiment, smoking pattern of the patient can be determined based on smoking history information that includes information such as but not limited to a number of smoked cigarettes, smoking urge and the like.

In an exemplary embodiment, a number of cigarettes consumed by the patient during a day can be monitored along with the time of the day at which the cigarette is consumed to determine the smoking pattern of the patient. Also, various triggers can be determined which lead to increase in the patient's urge for smoking.

Further, a machine learning-based technique can be used to estimate the patient's health based on the determined lifestyle pattern of the patient, the patient's lifestyle choices, dietary and exercise related preferences. Also, machine learning in healthcare can be used to analyze the health care records of the patients to suggest different data points and outcomes to provide timely risk scores, and precise allocation of the expert for the patient's treatment.

In an exemplary embodiment, personalized treatments can not only be more effective in pairing individual health with predictive analytics but is also beneficial for better disease assessment. Machine learning leverages the patient's medical history to help generate multiple personalized treatment options.

Further, above discussed parameters related to the patient's lifestyle, dietary pattern, and exercise regime along with health care plan recommended by an expert of a health care team can be used to compute a health score for the patient.

In one implementation, the patient's health score computation engine 212 may be configured to receive ratings in form of scores for each of a plurality of parameters related to the patient's lifestyle. In an embodiment, the parameters may be related to patient's lifestyle, dietary, exercise regime, and physiological behavioral, emotional and physiological aspects. In an embodiment, the patient may provide a score input corresponding to each of the plurality of parameters. In an embodiment, the score input may be in a range of 1-10, wherein a score of 1 may depict that the parameter being least applicable and score of 10 being most likely applicable to the patient.

In an exemplary embodiment, according to ayurveda, basic body constituency of the patient may be computed based on the principles of prakritis in Ayurveda. There are 3 main prakritis—kapha, vata, pita and a mixed set of prakritis that may be further computed based on the 3 main prakritis. In addition to the prakritis, there are two additional states of a user's body constituency—aama and agni. Therefore, the three prakritis and the two body constituencies form the basic output parameters defining a health status of a patient.

The present disclosure provides an improvement in the existing ways to find the five output parameters for the patient. The system 108 includes manually following a process to determine the five output parameters for a patient by an ayurvedic expert. The questionnaire may be manually vetted by an Ayurvedic expert and based on some rules and gives scores for each of the 5 parameters.

In the proposed disclosure, the machine learning based method may be data driven and such a method will take into account different combinations of the data. This is especially critical because the questionnaire includes of multiple sets of questions covering various aspects of the individual. Therefore, manually designing rules to cater to multiple combinations of the inputs is not accurate. The machine learning based method learns the rules from data and hence is more accurate.

In an embodiment, standard rules are applied as a human Ayurvedic expert determines the three prakritis and the body constituencies of a patient from a questionnaire. The proposed method includes creating an n-dimensional input vector x in the following manner using a format mentioned below in equation (1):

x={r₀, r₁, . . . , rᵢ, . . . , rₖ, cₖ₊₁, c₂, . . . , cⱼ, . . . cn−1} (1)

wherein rᵢ [symbol] R, and, 0≤i≤k
and cⱼ, C, and k+1≤j≤n−1;
wherein, the input vector x may be of dimension n and can comprise two sets of inputs. One set of inputs (represented by rᵢ in accordance to equation (1)) are real valued numbers that are continuous variables for the machine learning model that will be filled by the user as part of the questionnaire. The second set of inputs (represented by cᵢ in accordance to equation (1)) includes categorical variables that can have specific categorical values as provided by the Ayurvedic expert.

Based on the above inputted two sets of inputs, a new machine learning based model is designed. In the current implementation, an XGBoost based decision tree model is implemented. However, implementation can also include other machine learning based models such as but not limited to Random Forests, CatBoost (if inputs are to be considered as categorical variables only), Support Vector Machines and Deep learning based methods.

The machine learning based model is designed as a regressor model in the current implementation. The ground truth output labels includes ratings between a range of 1 to 3 for each of the 5 output variables—kapha, vata, pita, aama and agni.

An objective function of the model is designed as an L2 loss function as represented by equations (2) and (3) below:

$$\begin{matrix}L = \frac{1}{N} {\sum }_{\text{?}} ( Li ) & ( 2 ) \\ = \text{?} \sum {\text{?}}_{2} ) \\ \text{?} \text{indicates text missing or illegible when filed} & ( 3 )\end{matrix}$$

Where [symbol] is the ground truth output label, f is the machine learning model that maps an input x [symbol] to the output.

The method outputs a value between 1 and 3 for each of the 5 output nodes that indicate which pratrikiti the user is most likely to exhibit. In an embodiment, the objective function of the model may be designed as a loss function but not limited to L1 loss or Huber loss. Also, the same problem can also be formulated a classification problem with a log loss or softmax loss. Based on the score input provided by the patient, the health score computation engine 212 may generate a plurality of ratings associated with the patient's health status parameters. The health score computation engine 212 may query the database 120 to determine a mapping based on NLP AI methods.

In an embodiment, the plurality of ratings may depict a likelihood of a patient's having or showing one or more health conditions. In an exemplary scenario, the one or more health condition may include the three Prakritis as defined in accordance with Ayurveda, comprising kapha, vata and pita. The one or more health condition may include further include the body constituencies as defined in accordance with Ayurveda, comprising aama and agni.

In an embodiment, a first set of input parameters rᵢ associated with the entity 112-1 (e.g., the patient) are received. The first set of input parameters are indicative of one or more attributes of the entity 112-1 and are representative of one or more continuous variables. The one or more attributes of the entity may correspond to any or a combination of behavioral, emotional and physical characteristics of the entity 112-1. The represented one or more continuous variables may be real value numbers and may be provided as input to the first set of instructions, and wherein the first set of input parameters are representative of the one or more continuous variables and are received using questionnaire data provided by the entity 112-1. In an embodiment, the first set of instructions may include any of machine learning model, an XGBoost based decision tree model, and a random forest model.

### Second Input Parameters Receiving Engine 124

Further, a second set of input parameters are received from a second entity 114-1(e.g., health care expert) associated with the entity 112-1, the second set of input parameters being indicative of one or more health categories cⱼ where the one or more health categories are associated with the first set of input parameters of the entity. The health categories are representative of one or more categorical variables. The received first set of input parameters and the received second set of input parameters are analyzed to determine at least one of a health label for the entity 112-1. In an embodiment, the one or more continuous variables and the one or more categorical variables may be represented as an n-dimensional input vector x, where x={r₀, r₁, . . . , rᵢ, . . . , rₖ, cₖ₊₁, c₂, . . . , cⱼ, . . . , cₙ₋₁}, and where rᵢτ R, and 0<i<k, and cⱼτ C, and k+1≤j≤n−1 for recommending the food item to the entity.

### Input Parameters Analyzing Engine 126

Subsequently, a second set of instructions are analyzed to assign a health score for at least one of the health label for the entity 112-1. The health score is assigned based on a food item to be recommended. Upon the assigned health score being within a predefined threshold, the food item is recommended to the entity 112-1. In an embodiment, the execution of the second set of instructions and the first set of instructions is optimized using an L2 loss function. The L2 loss function may be represented as

$$L = \text{?} \sum \text{?} , = \frac{1}{N} {\sum }_{\text{?}} ( {( f ( {x}_{i} ) - {\hat{y}}_{i} )}_{2} ) , \\ \text{?} \text{indicates text missing or illegible when filed}$$

and where [symbol] is a ground truth output label, f is a machine learning model that maps an input to an output.

### Health Score Assigning Engine 128

In an embodiment, the output may indicate the health score to be assigned based on the food item to be recommended. The output may be a numerical value in a range of 1 to 3, where the output value of 1 may indicate a low health score, 2 may indicate a neutral health score, and 3 may indicate a high health score. In yet another embodiment, the health score may be updated when a change is determined in the received first set of input parameters and when one or more instructions are received from the second entity.

In an embodiment, the data collected from the patient's health record can further be refined by searching through the patient's health record, to identify the patient's data required for a particular or secondary use. As can be appreciated by one skilled in the art, machine learning techniques can be used to predict and improve potency of the patient's data. The machine learning can ingest the patient's data, draw parallels and conclusions across disparate data sets to provide refined data. The refined data can then be abstracted further by performing operations such as categorizing, coding, transforming, interpreting, summarizing, and calculating. Further, the abstracted data can be used in future for decision making.

In an exemplary embodiment, data abstraction can be done by reviewing the patient's health record information and abstracting (i.e., extracting) key data, which can be used further. As a next step, analytics can then be performed on the abstracted data to determine treatment plans so as to improve the patient's health.

In an embodiment, the analytics performed on the patient's health record can aid the system 108 to diagnose the patient's diseases, suggest treatments to the patient's history based on parameters like age, physiological symptoms, exercise regime, dietary pattern etc. The data analytics can generate a dynamic report based on the refined patient's health record. Examples of the reports may include charts, graphs, pivot tables (e.g., the axis of which may be selectable by the patient in real time), dashboards, etc. Further, other available data analytics tools that clearly depict the patient's health status based on the lifestyle pattern and/or the health record of the patient can be used.

### Food Item Recommendation Engine 130

Upon the assigned health score being within a predefined threshold, the food item is recommended to the entity. In one implementation, the plurality of ratings associated with the patient's health status parameters may be considered as an input to determine a meal plan for the entity. In an embodiment, the system 108 may implement a machine learning based model. In current implementation, the current model may be based on a decision tree model like but not limited to XGBoost based decision tree model, Random Forests, CatBoostSupport Vector Machines or Deep learning based methods.

In an embodiment, the decision tree model may be designed as a regressor model which may output ground truth outputs to depict the plurality of ratings associated with the patient's health status parameters. The plurality of ratings may depict a likelihood of a patient's having one or more health conditions that may be in form of ratings in a range of 1 to 3 for each of the output variables.

In an embodiment, an objective function of the model may be designed to use the following L2 loss function as represented above. In an embodiment, other loss functions like but not limited to L1 loss or Huber loss may also be used for the implementation of the decision tree model.

In an embodiment, the output of the data driven health consultation mechanism may be used as an input for providing personalized food recommendations. The patient 112 may be recommended personalized food items based on the determined patient's health status parameters, records and lifestyle information. For example, the patient can be provided food recommendations based on the patient's body weight, height, age, gender, physiological parameters, lifestyle parameters, exercise schedule, sleep pattern, etc. Also, the recommendations can be based on choices the patients make for various other factors like level of physical activity, food preferences, and many more.

In an exemplary embodiment, the patient may be provided meal options based on exercise regime, health conditions and health records. Further, a kind of meal taken by the patient at dinner time can also be used to suggest breakfast menu. Also, when the patient accidentally takes excess calories and may fail to maintain his/her health, suggestions related to diets such that impact of excess diet on the patient is provided. For example, the patient may be suggested intake of food/drink items with minimal to no sweeteners such as non-sugar beverages in case the patient is diabetic.

In an exemplary embodiment, when the patient is following a regular exercise regimen, the food recommendations can be such that adequate food and fluid is consumed before, during, and after exercise to help maintain blood glucose concentration. The food recommendations may ensure that the patient is well hydrated before exercise and should drink enough fluid during and after exercise to balance fluid losses. The food recommendations can be context aware recommendations based on parameters such as location information of the patient, resources available in a restaurant visited by the patient (e.g. as mentioned in menu), ingredients available at the patient's house. The ingredients at the patient's house can be inferred automatically from a grocery list of the patient using artificial intelligence techniques.

In an embodiment, suggestions related to recipes may be provided. The recommended meal options may be customized based on the one or more health conditions determined to be associated with the patient. For this, a natural language processing method to provide recipe recommendations may be used. Each recipe may be provided an effectiveness score to depict effectiveness of each recipe in curing or helping the patient with one or more health conditions.

In an embodiment, suggestions related to recipes of the meal options may be recommended to the patient 112 based on the effectives scores of each recipe in order to help the patient cure one or more health conditions determined to be associated with the patient 112. In an exemplary embodiment, the effectiveness score may be defined in accordance with Ayurveda.

In an embodiment, the patient may be provided a list of recipes as recommendation based on word embeddings generated in the database 120. The word embeddings maps words, text and phrases of recipes to Ayurvedic parameters defining five output parameters corresponding to the prakritis and body constituencies defined by Ayurveda. In this scenario, user has possible choices of food items such as ingredients or quantities, and the user's Ayurvedic doctor also gives possible recommendations. Using the text from these two inputs, the word embeddings will be searched to get possible recipes. The search is based on techniques like lowest distance measure to the centroids of output parameters.

The analysis based on querying of the database 120 using NLP AI based methods. In an embodiment, each recipe may include a time of preparation and a user rating depicting if the recipe has been tried and liked by users in the past. In an embodiment, the patient may select a recipe based on an effectiveness score, a user rating and time of preparation.

In an embodiment, the caregiver team can include experts such as doctors, nurses, nutritionist, technicians, and patient advocates (such as family members or social workers), who can aid in providing a health plan that includes suggestions related improvement in diet, sleep pattern, exercise regime, holistic medication and mental health of the patients.

In an exemplary embodiment, more than one expert of the caregiver team can be provided and mapped to the patient based on the patient's health-related conditions. In one example, each of the experts can be equipped to monitor the patient's specific health-related and lifestyle-related parameters (which includes the patient's dietary details, exercise regime, sleep pattern, smoking habit, etc.). The determination of the patient's information can then be used to select an expert from the caregiver team with specialization in handling cases similar to the patient's health record. The expert is then referred for future treatment and counseling of the patient.

In an exemplary embodiment, information about the caregiver team with experts onboard can be maintained in a knowledge base. The information can include, for example, the expert's communication conduct, receptiveness to outside influences, attitude to determine disease/symptoms/problems approach, decision making, explanation etiquette, relationship building, treatment tendencies, and/or lifestyle logistics. In addition to this the expert's specialty, subspecialty, qualifications, education, hospital affiliations, and/or awards can be maintained in the knowledge base.

In an exemplary embodiment, a numeric compatibility score can be calculated for each expert-patient pair. Higher numbers in the score can imply efficient synchronization between the expert and the patient, while lower numbers can portray synchronization discord between the expert and the patient (or vice-a-versa). Based on the compatibility score the expert can be recommended for the patient's treatment in future.

In an exemplary embodiment, selection of the expert from the caregiver team can be performed by selecting the expert based on his/her skills stored in the knowledge base and the determined patient's information. Further, the selection of the experts can be performed based on a causal probabilistic network such as a Bayesian network, fuzzy logic, a decision tree, a neural network or a self-organized map etc. Also, mapping of the expert to the patient for providing treatment can help in building the knowledge base over a period of time.

In an embodiment, the system 108 can implement artificial intelligence by using one or more processors that can be pre-programmed with computer readable instructions. The system 108 is an intelligent system and can include various machine learning trained models, deep learning models, artificial neural networks, fuzzy logic control algorithms, etc. The artificial intelligence can be implemented by the one or more processors 112 and memory 116. The processors 112 can dynamically update the computer readable instructions based on various learned and trained models.

The system 108 can also include an interface(s) 114. The interface(s) 114 may include a variety of interfaces, for example, interfaces for data input and output devices referred to as I/O devices, storage devices, and the like. The interface(s) 114 may facilitate communication of the system 108 with various devices coupled to the system 108. The interface(s) 114 may also provide a communication pathway for one or more components of the system 108. Examples of such components include, but are not limited to, processor(s) 112 and memory 116.

In an embodiment, the system 108 can provide an intelligent cloud-based platform that assists the experts of the caregiver team and the patients to collaborate and provide/receive health care treatment. The system 108 enables the patients to interact with the experts, of the caregiver team, from different domains/specialties based on the patient's preferences and requirements. The system 108 can aid the patients with personalized recommendations based on the patient's lifestyle preferences and provide health care recommendations via the experts based on the stored patient's health record.

In an embodiment, the system 108 assists the caregiver team and the patients in multiple ways by aiding the patients to interact with members of the caregiver team. The members of the caregiver team can be experts from different domains or specialties. Each of the patients can be aligned with a member of the caregiver team based on the patient's health diagnosis, and preferences and requirements based on the patient's health. The system 108 can facilitate to provide personalized recommendations to the patients. Further, the caregiver team can provide data driven consultations based on the lifestyle activities and the health records of the patients.

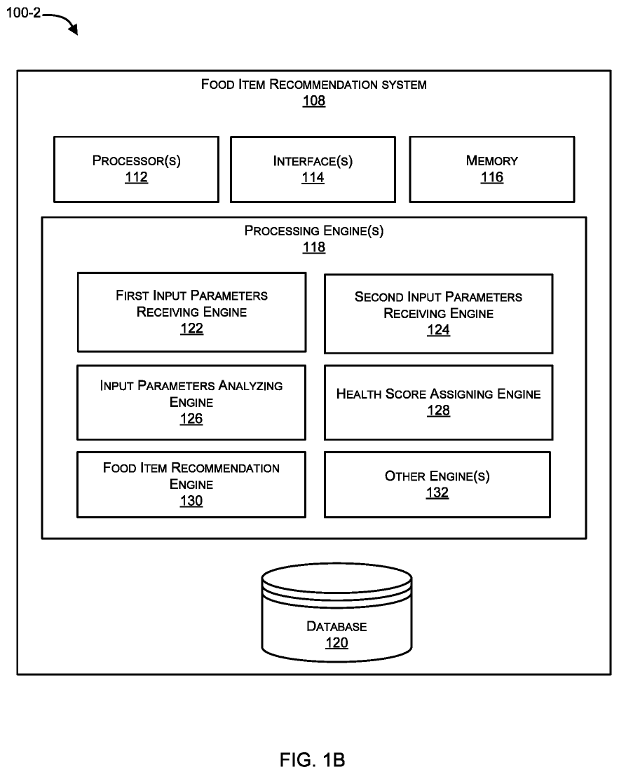

FIG. 2A illustrates at 200-1 a high level architecture of a natural language processing, based on recipe score recommender in accordance with an embodiment of the present disclosure.

With reference to FIG. 2A, at block 202, an input is taken for each recipe corresponding to but not limited to recipe's name, ingredients, quantities of ingredients and the process of cooking the recipe etc. In an embodiment, each ingredient may be given a rating based on its general effect in curing or aggravating one or more pre-defined health conditions. In an embodiment, the ratings are pre-defined as mapping in a database 120. In an embodiment, the ratings are generated based on the various machine learning methods disclosed herein. In an embodiment, an ingredient may be rated between 1 to 3 corresponding to impact of the recipe on the health of the entity, where 1 may represent that the ingredient may help in decreasing a health condition and 3 may represent that the ingredient may help in aggravating a health condition. At, block 204, word embedding is performed based on techniques such as but not limited to Bag of words (BOW), Word2Vec, etc. to generate a vector based mapping based on words corresponding to the input variables and their associated ratings. At block 206, a deep neural network backbone architecture is implemented, to extract features from the words corresponding to the input variables and the features may be used by the architecture. The extracted features may encode the input variables into a feature representation. In an embodiment, the architecture may include models but not limited to resnet, xception, mobilenet etc. At block 208, a regression model is implemented which may predict a score that corresponds to one or more health conditions for each of a health plan represented at block 210-1, 210, . . . 210-5 for each recipe as an output. In an embodiment, the one or more health plan may include the three Prakritis and two body constituencies as defined in accordance with Ayurveda.

FIG. 2B illustrates at 200-2 determining word embeddings obtained from deep neural network in accordance with an embodiment of the present disclosure.

With reference to FIG. 2B, inputs such as attributes and preferences of an entity (e.g., a patient) at block 212 and expert inputs and associated health labels are determined at block 214. Further, at block 216, recipe details of a recipe to be evaluated are inputted. At block 218, a processing is performed taking as input the output of the block 212, 214, and 216. A natural language processing is performed at block 218 to determine a health score for the entity upon consumption of the recipe. The processing may include measuring a lowest distance to one or more centroids corresponding to one or more output parameters from one or more input parameters. In an embodiment, the output parameter may correspond to a target health score. In an embodiment, a target health score may be created based on determined health status and an expert input. At block 220, a recipe recommendation is generated for the recipe under consideration to be evaluated, which may comprise of but not limited to one or more recipes. Each recipe that is suggested may be based on determined effectiveness score, a user rating and a time of preparation for the recipe.

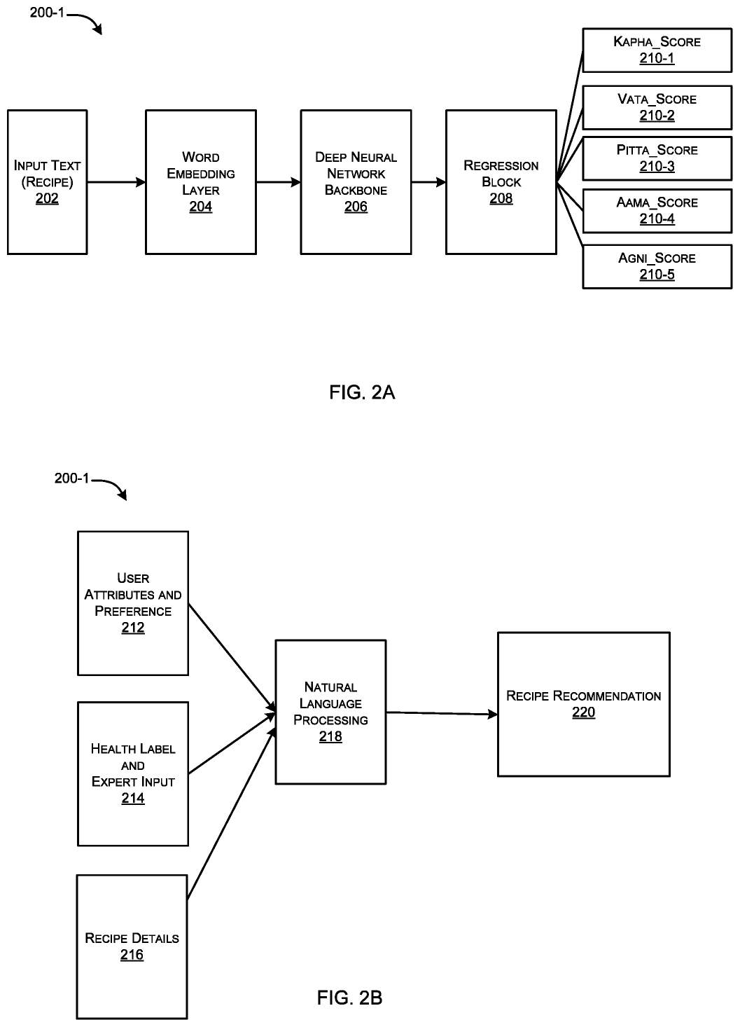

FIG. 3 illustrates a framework 300 of the food item recommendation system, in accordance with an embodiment of the present disclosure.

In an embodiment, at block 302 a mobile sensor can be provided in the wearable device and/or a health monitoring device, which is used to capture the patient's health-related information. The health-related information is captured by determining parameters such as age, weight, gender, eating/drinking pattern, exercise regime etc. of the patient. The monitoring devices can be such as but not limited to the smart watch, wristband, smart phone, meditation band, smart clothing, or other device including sensors capable of capturing the patient's activity data and vital sign data (e.g., gyroscope, accelerometer, magnetometer, infrared sensor, camera, microphone, gas sensor, photo-detector, etc). At block 304, the pattern of the patient's lifestyle is detected, based on the physiological parameters of the patient, exercise regime and eating/drinking habits of the patient, etc.

At block 306, the patient's lifestyle related information is logged and is stored in the knowledge base to be used for future reference. In an embodiment, the lifestyle preference of the patient can be determined and estimated at block 308 through the detected patient's lifestyle at block 304 and the logged information related to the tracked patient's lifestyle at block 306. At block 308, the patient's lifestyle preference estimation can be determined based on parameters such as food items, physical exercise, vitals, etc. which can be used to model the patient's lifestyle. The modeling can be done either using univariate or multivariate distributions or probabilistic modeling or generative modeling or other methods such as linear regression or non-linear regression functions. The model can be used to provide a probabilistic score for the patient's lifestyle.

In an embodiment, the expert can have access to the patient's data via the patient's health care data log at block 306 that is maintained while collection of the patient's data. The data logs can be used to create a variety of distribution curves. The distribution curves can provide joint probabilities between different lifestyle parameters. For example, given diet inputs from the patient, probability density curves for timing of food consumption can be generated, i.e., p(time|food). The distribution curves can further be used to determine a probability of eating specific kinds of foods represented as p(food_type|time). Such detailed data-driven dashboards can then be provided for each of the patients to the expert. The experts can use the dashboards to give an informed consultation. Also, the generated probabilities can be computed for different time periods, for the expert to choose.

In an embodiment, at block 306 the patient's lifestyle logging can log information related to the lifestyle parameters of the patient such as food items consumed, physical exercise, vitals, etc. The lifestyle parameters can be used to create generative probabilistic distributions. The generative probabilistic distributions can be represented either as a univariate expression or a multivariate expression using probability distributions. As an example, the generative probabilistic distributions can generate probability distributions in the following manner using following conditional probability equation:

$$\hat{y} = argmax {p}^{'} ( {C}_{k} ) {\prod }_{i = 1}^{n} {p}^{'} ( {x}_{i} \vert {C}_{k} ) \\ k ∈ \{ 1 , … , K \} \qquad ( 4 )$$

and where p(y) is the probability score of a given variable y,
xᵢ is a probability of the patient having a particular food type, and
Cₖ is time period during which the patient is having the particular food type.
For example, probability of a patient having a type of food xᵢ during a time period Cₖ can be obtained by using the above conditional probability equation.

Further, the probability distributions along with the distributions from the health care plan features can be compared using techniques such as KL divergence or cosine distance function. Probability distributions along with the distributions from the health care are combined into cosine distance function to determine the health score as represented below:

Health Care Score−w^{T}p (5)

where, w^{T} is a feature vector representing the distribution of the health care plan, and

p is a feature vector from distributions of lifestyle.

For example, in the above equation, w can be the feature vector representing the distribution of health care plan and p is the feature vector from the distributions of lifestyle.

Based on information determined at block 304 and at block 306, health analytics of the patient's health record is computed at block 310.

In an embodiment, the patient's health care data collection can be followed by data abstraction, which involves extracting higher-level features of the patient's data. The abstraction can be performed by using techniques such as but limited to one-to-one mapping, one-to-many mapping, look-up-table mapping or bag-of-words based techniques or linear regression and quantization or similar techniques.

In an embodiment, the patient's health care data can be collected to perform the patient's health analytics at block 310. The health care data includes tracking the patient's vitals such as blood pressure, sugar levels, etc. Also, the lifestyle parameters like food habits, physical activity, and sleep activity can be determined. The collected data is abstracted further for performing data analytics and tracking.

In an embodiment, the data analytics on the abstracted patient data can be performed at block 310. The data analytics can be performed by modeling the data using probabilistic models or generative models or Bayesian modeling or conditional probabilities. The modeling can include univariate distributions or multi-variate distributions. The distributions can be either Gaussian models for each individual parameter or mixture of Gaussians to cater for the multi-variate distributions. The distributions can be non-Gaussian like a Poisson distribution etc.

In an embodiment, at block 310 the data analytics can be performed and can enable initiating conditional probabilities for each of the data parameter against another such as p(time|food). The Bayesian modeling can generate different combinations of the parameters for tracking. These parameters can be tracked over time and predictions can be generated. The parameter tracking can involve use of raw data/parameters logging and data filtering using techniques such as averaging filters (like Gaussian filters, box filters etc.) and median filtering (for non-integer based parameters).

At block 312, a health care plan is suggested and provided by the experts of the caregiver team to the patients. The suggested health care plan is based on the determined patient's lifestyle and physiological information. Also, the health care plan suggested by the experts at 312 can be used to perform the patient's health analytics at block 310.

In an embodiment, the health of the patient can be quantified as a health care score using a mathematical model. The mathematical model uses a mathematical function to compute the health score in a form of y=f (a, b, c, . . . ) where y is the health score and a, b, c, . . . are the different input parameters that determine the health score. The function f( ) can either be a linear model, a quadratic model, a logarithmic model or any non-linear model. The choice of the model can be made either empirically by choosing one model that is suitable to the system or by using the model that fits in the patient centric data.

In an embodiment, the health care plan can be codified into a vector format, which can then be used as an input into the function f( ). Codification of the health care plan can be done by different means, for example by using a natural language processing algorithms (NLP) like word2vec. Other techniques such as bag of words, histogram of words, etc. can also be used to code the health care plan. Codification format can be a one-hot coding or a multi-label code.

In an embodiment, the health care score can then be determined for the patient based on both the determined lifestyle pattern of the patient and the suggested health care plan by the expert. Given the health care plan in the codified form and the lifestyle patterns of the patient, the function f( ) can combine different parameters into the health care score. It is to be appreciated that a combination of the models can be created in a cascaded manner to generate the patient's health care score. Further, if the expert's health care plan is provided, the system 108 can use word2vec algorithm to code the health care plan to a one-hot coded health care plan. The one hot coded health care plan can be represented as health care plan-> word2vec (health care plan)-> one-hot coding->health care plan features (w).

In an embodiment, based on the lifestyle preference estimation at block 308 and the health plan recommended by the experts at block 312, a personalized lifestyle recommendation is provided to the patients at block 314, which can pertain to providing recommendations to the patients related to diet, exercise regime, and/or suggestions related to shopping of food items etc.

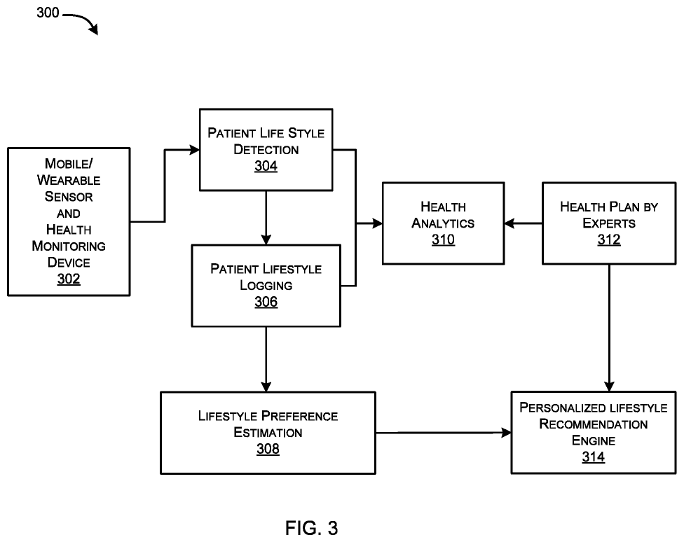

FIG. 4 illustrates a detailed framework 400 of the food item recommendation system, in accordance with an embodiment of the present disclosure.

In an embodiment, at block 402 an automated lifestyle data gathering related to the patient's lifestyle is performed. The data gathering can be performed at block 404 via the wearable devices such as but not limited to the smart watches, smart bands, smart rings, meditation band, and the like. The data gathering via the wearable devices can be performed by automatically recognizing the patient's activity at block 406. For example, the patient's activity can be recognized by detecting eating/drinking patterns of the patient at block 408, which can be performed by using the IMU sensors, heart rate detection sensors, and GPS. At block 410, sleep pattern detection of the patient can be performed by using any of the IMU sensors, monitoring microphone and screen activity of the device. At block 412, the exercise pattern of the patient can be determined via the IMU sensors. Further, at block 414 other activities of the patient such as smoking can be determined by using the IMU sensors. The recognitions and determinations at the block 408, 410, 412 and 414 can be executed at particular intervals of timestamp. Furthermore, at block 416 the recognitions and determinations performed at the block 408, 410, 412 and 414 are collected and maintained in an activity data log that contains all information related to the sleeping, exercising and eating patterns of the patient.

In an embodiment, at block 418, the data related to the patient's lifestyle can be gathered using the mobile devices such as a smart phone, tablet or any other computing device. At block 420, mobile devices can be used to determine details of the patient's activity. At block 422, the patient's activity can be determined by capturing and tagging the eatable items to be consumed by the patient, via the mobile device's camera. Further, at block 424, the patient's sleep quality detection can be performed via the IMU sensors, monitoring microphone and screen activity of the mobile device. At block 426, the patient's exercise routine/yoga asanas detection can be performed using the mobile device's camera. The information determined at block 422, 424, and 426 can be further tagged and fed as input to a block 428, where details of the patient's activity are logged. The patient's activity information can include the eating patterns of the patient, exercise regime/yoga asana practiced by the patients and the patient's sleep pattern. In an embodiment, the patient information detected at block 404 via the wearable's devices and at block 418 via the mobile devices can be exchanged for better understanding of the patient's lifestyle routine.

In an embodiment, at block 430 the patient's physiological information can be determined via the use of health monitoring devices. At block 432, the health monitoring devices can be used to determine the patient's health vitals such as but not limited to blood pressure, sugar levels, weight, heart rate etc. Additionally, the patient can input his/her medical history information at block 434.

In an embodiment, activity log determined at block 416, details of eating patterns, exercise regime and sleep pattern of the patient determined at block 428, patient's health vitals determined at block 432 and medical history information of the patient determined at block 434 are fed as input to a data analytics block 438. Analytics and trends for one of the patients can be determined at block 440 and at block 442 global trends across the multiple patients can be determined. Further at block 444, automated anomaly detection in the patient's health records can be performed. Furthermore, at block 446 a co-relation between health metrics and lifestyle of the patients can be established. In addition to this, at block 448 a health care score can be computed for the patient and assigned to the patient under consideration.

In an embodiment, at block 448, the health care score may include a plurality ratings associated with the patient's health status parameters. In an embodiment, the plurality of ratings may depict a likelihood of a patient's having or showing one or more health conditions. In an exemplary scenario, the one or more health condition may include the three Prakritis as defined in accordance with Ayurveda, comprising kapha, vata and pita. The one or more health condition may include further include the body constituencies as defined in accordance with Ayurveda, comprising aama and agni.

In an embodiment, the data analytics performed at block 438 can be converted and presented in form of dashboards to consultants that act as an intermediary between the experts and the patients. Also, the consultants can provide video, audio, or text transcripts at block 436.

As an example, block 452 shows a health care plan as prepared by the experts of the health care team for the patient to follow. The health care plan at block 454 includes diet plan for the patient. The feeds for the dietary plan can be avoided and a referral can be added to provide holistic diet experts. At block 456, natural medicines/supplements can be suggested and refer to providing holistic diet experts. Further, at block 458, plans related to exercise/yoga asanas can be suggested, which include suggestions related to yoga asanas, referral to yoga experts. Furthermore, at block 460 tips on how to maintain the patient's mental health can be provided, which can include suggesting breathing exercises, and meditation practices. Further, the block 460 includes referring a meditation expert to the patients. At block 462, other sleep and lifestyle related advice can be provided to the patient. The health care plan as suggested by the experts at block 452 can be shared with the data analytics block 438.

In an embodiment, the determined patient's lifestyle information at block 402, the analyzed data determined at data analytics block 438 and the health care plan as suggested by the experts at block 452 can then be used to calculate the health care score at block 450. The health care score at block 450 can be determined at scheduled intervals such as daily, weekly or monthly.

In an embodiment, at block 464, the activity log information as determined at block 416 and the detail of the activity pattern of the patient related to food eating pattern, exercise regime and sleep pattern of the patient determined at block 428 can be collated to determine personal preference of the patient. Further, at block 466, one or more models can be used to learn and understand the patient's preferences. The models can be such as linear model, quadratic model, logarithmic model and like. In an embodiment, models may be decision tree models like but not limited to XGBoost based decision tree model, Random Forests, CatBoostSupport Vector Machines or Deep learning based methods. The preferences can be related to determining food preferences such as cuisine types and habits of the patients at block 468. At block 470, the models can be used to determine exercise preferences for the patient such as whether the patient prefers outdoor/indoor exercise, favorite sport, ease of doing asanas etc. In addition, at block 472 the models can be used to determine the sleep habits/pattern and other lifestyle preferences of the patient.

In an embodiment, the determined food preferences of the patients at block 468, exercise preferences for the patient at block 470, and the sleep habits/pattern of the patient at block 472 can be shared as input to a personalized lifestyle assistance block 474. The personalized lifestyle assistance block 474 can also receive the health care plan as suggested by the experts at block 452 as input.

In an embodiment, the personalized lifestyle assistance block 474 may also include recipe recommendation which may be generated by a food recommendation engine 480 (explained later). The recipe recommendation is determined based on but not limited to the food preferences, lifestyle preferences, healthcare score determined and expert health plan input for the patient.

In an embodiment, the personalized lifestyle assistance block 474 can include a food recommendation engine 480, a yoga tracker and assistant 482, and a shopping assistant 484. The food recommendation engine 480 can suggest about recipes to be cooked at home at block 486, food to be ordered in restaurants at block 492. The yoga tracker and assistant at block 482 can track the patient's asanas at block 488, and provide real time asanas suggestions to the patients at block 494. Further, the shopping assistant 484 can provide various product buying suggestions to the patients at block 490.

In an embodiment, the food recommendation engine 480 can include providing food recommendations based on the patient's preferences, the patient's dietary patterns and the patient's health care plan as recommended by the experts. The food recommendations can also be provided based on the patient's location and time to determine where and when the patient is.

In an embodiment, the food recommendation can be modeled using a complex function of the form y=f(a, b, c, . . . ) where f is a function that is parameterized by variables a, b, c etc. These variables can correspond to outputs from individual functions. Therefore the overall formulation can be represented as:

y=f(φ(a),Ψ(b),ζ(a,b,c), . . . ) (6)
where, y is output of the recommendation.

In context of an example, each of the input functions can be formulated in a following manner. A first function is a function that can generate the patient's dietary timing distributions, which can include a uni-variate modeling of the patient's eating pattern by taking in input when the patient chose to eat a meal. Therefore, the function is a function of time f(t). The modeling can include either multiple Gaussian distributions or Poisson distributions or any such distributions based on the data. Further, graph fitting techniques and clustering techniques such as k-means clustering can be used to determine the appropriate distribution and its statistical means and variances.

In an embodiment, formulation of the patient's dietary preferences in terms of what the patient consumes can be determined. The patient's dietary preferences can be modeled using univariate or multivariate bayesian models, or via clustering methods or nearest neighbor based methods such as k-nearest neighbor model (KNN).

In an embodiment, the food to be ordered in restaurants by the patients can be determined at block 492, which can be performed by using techniques such as but not limited to deep neural networks that parse texts in menus to map food items in restaurants to specific categories. The categorization of the food items can involve using a natural language processing (NLP) algorithm, to first parse the text followed by a deep neural network to perform a multi-class classification and tagging. Further, inputs of the food items can be provided by using a camera of the mobile device that can take a picture of the menu, and then parse the text to determine the menu's content items.

In an embodiment, the personalized lifestyle assistance at block 474 also takes as input the heath care plan as suggested by the expert at 452. The health care plan for the patient can be codified into a vector format which can then be used as an input into the function f( ). The codification can be done by different means, for example by using a natural language processing algorithm (NLP) like word2vec. Other methods like bag of words, histogram of words, etc. can also be used to code the health care plan. The codification format can be a one-hot coding or a multi-label code. In an embodiment, alerts 476 and reminders 478 can be provided to assist and motivate the patients to maintain a healthy lifestyle.

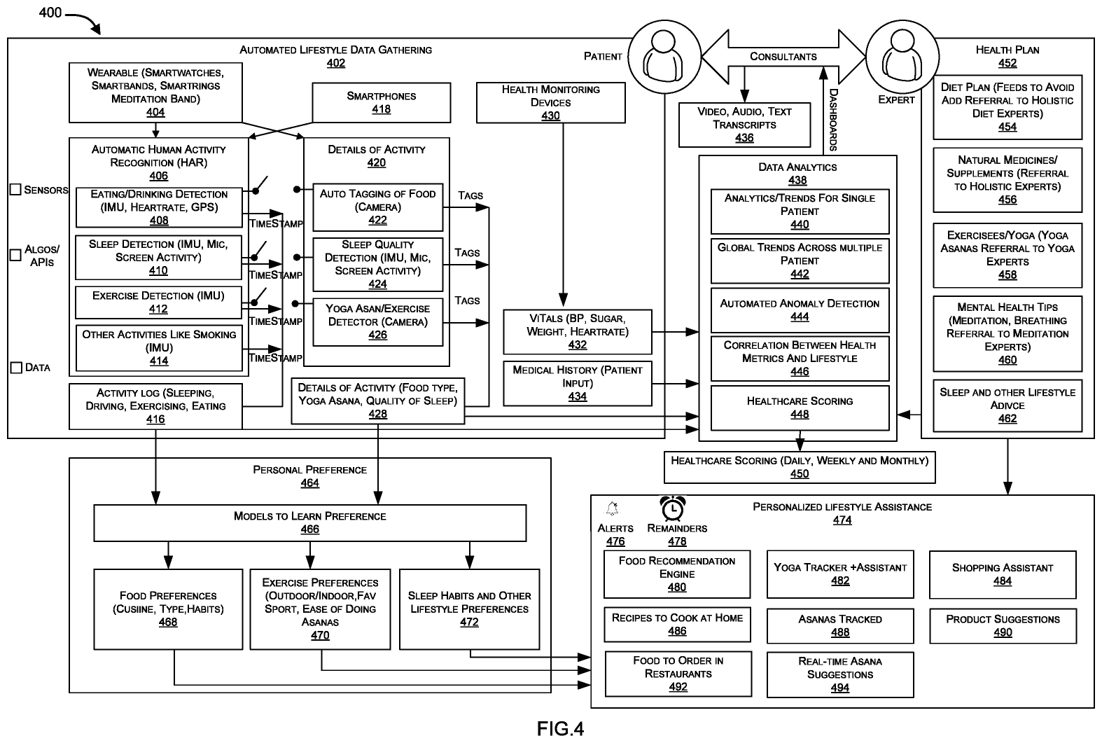

FIG. 5 illustrates at 500 a deep learning-based recommendation engine in the food item recommendation system in accordance with an embodiment of the present disclosure.

In an embodiment, recommendations based on the determined lifestyle pattern of the patient are provided by the system. The system can provide the recommendations related to such as a diet plan, natural medicine/supplements, exercises/yoga asanas, mental health maintenance tips, and sleep and other lifestyle advice. The recommendations can be provided using artificial intelligence (AI) techniques, which can be implemented using a deep-learning based recommendation engine.

In an embodiment, at block 502, a deep learning-based recommendation engine can be used such as but limited to a collaboration recommendation engine, a content-based recommendation engine, a hybrid engine and the like. In the recommendation engine, inputs can be taken as a combination of multiple aspects of the patient's activity as well as global pool of the patient's activity and choices. The first part of the input can be the patient's own activity and includes determining the patient's own personal preferences, the patient's past usage statistics, the patient's ratings and reviews and the patient's lifestyle activity requirements. The second part of the input can be a global pool of the patient's activity, which can also include a choice of the experts made by the other patients. A combination of both of these inputs can be used in a deep learning based recommendation system to recommend a combination of the health care experts to the patient.

In an embodiment, at block 504 the recommendation engine can provide recommendations related to diet, exercise regime, medication, sleep, and mental health for the patient. The recommendations can be provided by using a neural building block. Further, at block 508 the determination of the experts can be done using various techniques such as but not limited to using a convolutional neural network (CNN), recurrent neural networks (RNN), autoencoders (AE), variational autoencoders (VAE), etc.

In an embodiment, at block 506 the recommendation engine can provide recommendations for improving the patient's health parameters. Further, at block 510 the recommendations to be provided can be selected by using various techniques such as but not limited to using a combination of the RNN and the CNN models, a combination of AE and CNN models, and RNN and AE models, etc.

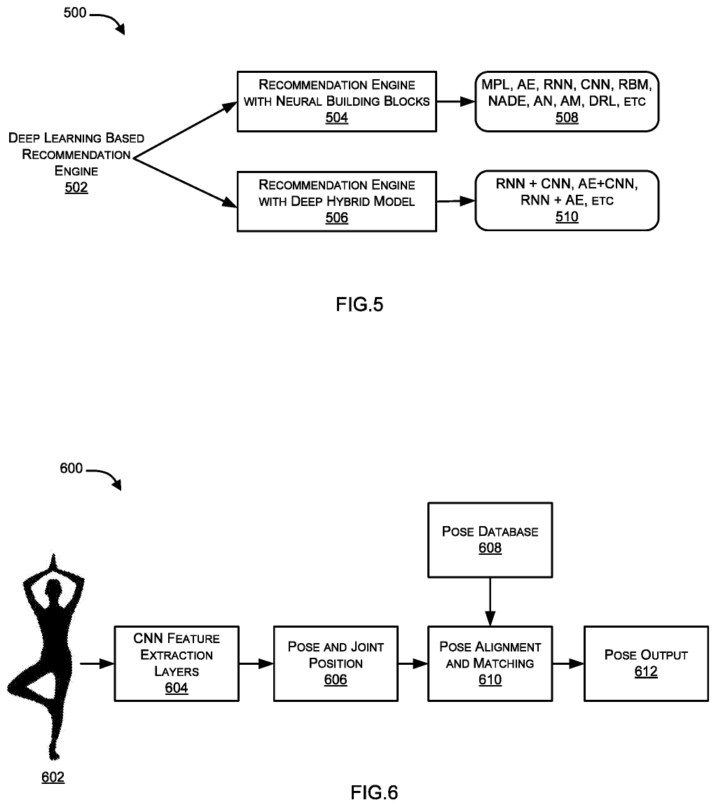

FIG. 6 illustrates at 600 a 3D joint estimation technique for posture determination of a patient in accordance with an embodiment of the present disclosure.

As an example, a deep learning/machine learning-based model for assisting the patients with yoga practice is illustrated. In an embodiment, assisting the patients with their yoga practice requires detecting yoga posture of the patient at block 602 using the sensors. The sensors can include cameras and/or accelerometers, gyroscopes. The camera used in the sensors can be either a monocular or a stereo camera so that 3D data of the patient's body can be captured.

In an embodiment, at block 604 the yoga postures can be detected using techniques such as but limited to a traditional computer vision based algorithms such as by using support vector machines (SVMs) or aggregated channel features (ACFs), or using deep learning networks such as Residual networks (ResNets), densenet, inception networks and like. Feature extraction can be performed by using the networks like resnet or densenet, and can be used to train a regressor, which can generate relative positions of joints of the patient's body. Further, at block 604, the determined input image can be represented as CNN layers, which can be used to detect the patient's yoga pose. Additionally, the patient's yoga practice can be logged and can be used to set personalized yoga goals for the patient.

The deep learning networks can be either end-to-end networks where the yoga postures can be detected using visual sensor data, or by using the deep learning architectures that can be used for feature extraction from the captured yoga postures of the patient. The features can further be processed for classification using different classifiers like a softmax classifier. For detection of the postures a set of non-visual sensors can be used and the classification can be performed by using the classifiers like Support Vector Machine (SVM) and XGBoost along with a visual data based classification, The features can either be raw signals from an accelerometer that are used to determine acceleration in x, y and z directions, or by using a combination of signals like magnitude of the acceleration.

In an embodiment, at block 606 different camera positions can be used to estimate 3D pose and joints position of the yoga practitioner by using various 3D joint estimation techniques. The 3D joint estimation techniques can be used that involves using features from deep learning architecture to determine joint probabilities between vertices of edges of stick diagrams to represent the yoga practitioner. The determined stick diagrams can be rotated to a reference frame and further classified. At block 608, prior information of the 3D image is determined from a pose database. In an embodiment, the determined prior information of the 3D image from the pose database at 608 and the pose and joint position estimation at block 606 are considered collectively to calculate pose alignment and matching for the practitioner at block 610. A resultant post output can be then obtained at block 612.

In an embodiment, the system 108 facilitates empowering the patients to receive health care recommendations from an expert or a set of experts of the caregiver team and helps the patients to avoid looking into the information overload on Internet. In addition, the information provided to the patients is personalized, based on the patient's lifestyle patterns, medical conditions and other dietary and exercise pattern information.

In an embodiment, use of analytics on the patient's data leads to better care of the patient and lower costs for the patients. The analytics enables making better decisions and allows for providing a personalized health care plan for each patient.

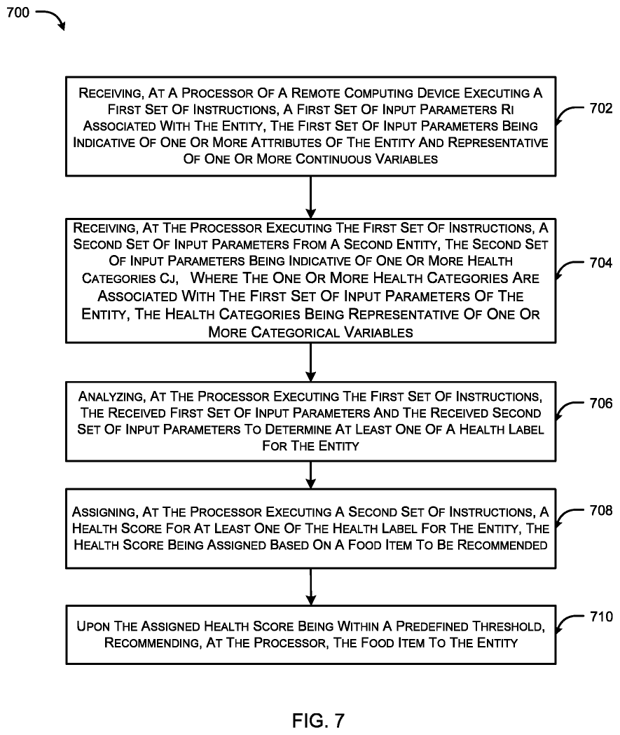

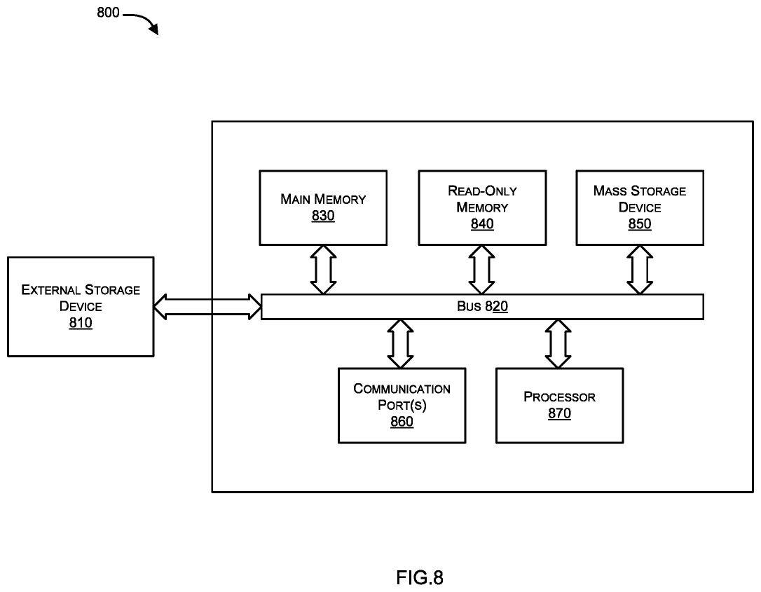

FIG. 7 is a flow diagram 700 illustrating a food item recommendation processing in accordance with an embodiment of the present invention. The process described with reference to FIG. 7 may be implemented in the form of executable instructions stored on a machine readable medium and executed by a processing resource (e.g., a microcontroller, a microprocessor, central processing unit core(s), an application-specific integrated circuit (ASIC), a field programmable gate array (FPGA), and the like) and/or in the form of other types of electronic circuitry. For example, this processing may be performed by one or more computer systems of various forms, such as the computer system 800 described with reference to FIG. 8 below.

Embodiments, described herein seek to, generate food item recommendations for an entity. At block 702, a first set of instructions are executed to receive a first set of input parameters ri associated with the entity. The first set of input parameters rᵢ are indicative of one or more attributes of the entity and representative of one or more continuous variables. At block 704, a second set of input parameters area received from a second entity, the second set of input parameters are indicative of one or more health categories cj, where the one or more health categories are associated with the first set of input parameters of the entity, the health categories being representative of one or more categorical variables. At block 706, the received first set of input parameters and the received second set of input parameters are analyzed to determine at least one of a health label for the entity. Further, at block 708, a health score is assigned for at least one of the health label for the entity. The health score is assigned based on a food item to be recommended. Upon, the assigned health score lying within a predefined threshold, at block 710 recommending the food item to the entity.

FIG. 8 is an exemplary computer system in which or with which embodiments of the present invention may be utilized. As shown in FIG. 8, computer system includes an external storage device 810, a bus 820, a main memory 830, a read only memory 840, a mass storage device 850, a communication port 860, and a processor 870. Computer system may represent some portion of the personalized patient health care recommendation system 108.

Those skilled in the art will appreciate that computer system 800 may include more than one processor 870 and communication ports 860. Examples of processor 870 include, but are not limited to, an Intel® Itanium® or Itanium 2 processor(s), or AMD® Opteron® or Athlon MP® processor(s), Motorola® lines of processors, FortiSOC™ system on a chip processors or other future processors. Processor 870 may include various modules associated with embodiments of the present invention.

Communication port 860 can be any of an RS-232 port for use with a modem based dialup connection, a 10/100 Ethernet port, a Gigabit or 10 Gigabit port using copper or fiber, a serial port, a parallel port, or other existing or future ports. Communication port 860 may be chosen depending on a network, such a Local Area Network (LAN), Wide Area Network (WAN), or any network to which computer system connects.

Memory 830 can be Random Access Memory (RAM), or any other dynamic storage device commonly known in the art. Read only memory 840 can be any static storage device(s) e.g., but not limited to, a Programmable Read Only Memory (PROM) chips for storing static information e.g. start-up or BIOS instructions for processor 870.

Mass storage 850 may be any current or future mass storage solution, which can be used to store information and/or instructions. Exemplary mass storage solutions include, but are not limited to, Parallel Advanced Technology Attachment (PATA) or Serial Advanced Technology Attachment (SATA) hard disk drives or solid-state drives (internal or external, e.g., having Universal Serial Bus (USB) and/or Firewire interfaces), e.g. those available from Seagate (e.g., the Seagate Barracuda 7200 family) or Hitachi (e.g., the Hitachi Deskstar 7K1000), one or more optical discs, Redundant Array of Independent Disks (RAID) storage, e.g. an array of disks (e.g., SATA arrays), available from various vendors including Dot Hill Systems Corp., LaCie, Nexsan Technologies, Inc. and Enhance Technology, Inc.

Bus 820 communicatively couples processor(s) 870 with the other memory, storage and communication blocks. Bus 820 can be, e.g. a Peripheral Component Interconnect (PCI)/PCI Extended (PCI-X) bus, Small Computer System Interface (SCSI), USB or the like, for connecting expansion cards, drives and other subsystems as well as other buses, such a front side bus (FSB), which connects processor 870 to software system.

Optionally, operator and administrative interfaces, e.g. a display, keyboard, and a cursor control device, may also be coupled to bus 820 to support direct operator interaction with computer system. Other operator and administrative interfaces can be provided through network connections connected through communication port 860. External storage device 810 can be any kind of external hard-drives, floppy drives, IOMEGA® Zip Drives, Compact Disc—Read Only Memory (CD-ROM), Compact Disc—Re-Writable (CD-RW), Digital Video Disk—Read Only Memory (DVD-ROM). Components described above are meant only to exemplify various possibilities. In no way should the aforementioned exemplary computer system limit the scope of the present disclosure.

While the foregoing describes various embodiments of the invention, other and further embodiments of the invention may be devised without departing from the basic scope thereof. The scope of the invention is determined by the claims that follow. The invention is not limited to the described embodiments, versions or examples, which are included to enable a person having ordinary skill in the art to make and use the invention when combined with information and knowledge available to the person having ordinary skill in the art.

1. A method for recommending a food item to an entity, said method comprising:
receiving, at a processor of a remote computing device executing a first set of instructions, a first set of input parameters rᵢ associated with the entity, the first set of input parameters being indicative of one or more attributes of the entity and representative of one or more continuous variables;
receiving, at the processor executing the first set of instructions, a second set of input parameters from a second entity, the second set of input parameters being indicative of one or more health categories cⱼ, where the one or more health categories are associated with the first set of input parameters of the entity, the health categories being representative of one or more categorical variables;
analyzing, at the processor executing the first set of instructions, the received first set of input parameters and the received second set of input parameters to determine at least one of a health label for the entity;
assigning, at the processor executing a second set of instructions, a health score for at least one of the health label for the entity, the health score being assigned based on a food item to be recommended; and
upon the assigned health score being within a predefined threshold, recommending, at the processor, the food item to the entity.

2. The method of claim 1, wherein the represented one or more continuous variables are real value numbers and are provided as input to the first set of instructions, and wherein the first set of input parameters representative of the one or more continuous variables are received using questionnaire data provided by the entity.

3. The method of claim 1, wherein the method further comprises:
extracting, at the processor, a sequence of words from the food item to be recommend to the entity;
determining, at the processor, a plurality of similar words based on the extracted sequence of words using a third set of instructions, the determined plurality of similar words being indicative of the food item to be recommended to the entity, and wherein executing the third set of instructions to map the food item to be recommended to at least one of the health label; and
receiving, at the processor, the health score for at least one of the health label, wherein upon the received health score being within the predefined threshold, recommending the food item to the entity.

4. The method of claim 3, wherein the determined plurality of similar words comprises at least one of a misspelling, synonym, abbreviation, metonym, synecdoche, metalepsis, kenning, or acronym associated with the extracted sequence of words.

5. The method of claim 3, wherein the method further comprises determining, at the processor, a difference level between the extracted sequence of words and the determined plurality of similar words, and if the difference level between the extracted sequence of words and the at least one of determined plurality of similar words is below a threshold value considering the at least one of determined plurality of similar words as a closest match to the extracted sequence of words.

6. The method of claim 1, wherein upon the determined plurality of similar words being indicative of the food item to be recommend to the entity and having being mapped to at least one of the health label and having the received health score lying within the predefined threshold, storing, at the processor, the determined plurality of similar words in a dataset, wherein each of the determined plurality of similar words are indicative of the food item to be recommend to the entity and is mapped to at least one of the health label.

7. The method of claim 1, wherein the first set of instructions comprises any of machine learning model, an XGBoost based decision tree model, and a random forest model.

8. The method of claim 1, wherein the execution of the second set of instructions and the first set of instructions is optimized using an L2 loss function, where the L2 loss function is represented as L=_{N}¹Σᵢ(Li), =_{N}¹Σᵢ((f(xᵢ) ŷᵢ)²), and where ŷᵢ is a ground truth output label, f(xᵢ) is a machine learning model that maps an input xᵢ to an output, the output being indicative of the health score to be assigned based on the food item to be recommended.

9. The method of claim 8, wherein the output is a numerical value in a range of 1 to 3.

10. The method of claim 8, wherein the output value of 1 is indicative of a low health score, 2 is indicative of a neutral health score, and 3 is indicative of a high health score.

11. The method of claim 1, wherein the third set of instructions comprises any of a neural network language model, and natural language processing mechanism.

12. The method of claim 1, wherein the one or more continuous variables and the one or more categorical variables are represented as an n-dimensional input vector x, where x={r₀, r₁, . . . , rᵢ, . . . , rₖ, cₖ₊₁, c₂, . . . , cⱼ, . . . , cₙ₋₁}, and where rᵢ∈R, and 0≤i≤k, and cⱼ∈C, and k+1≤j≤n−1, and the variables are indicative of an association of the entity to at least one of the health label.

13. The method of claim 1, wherein the method further comprises:
receiving, at the processor, a set of entity preferences, the health label for the entity, and at least one of an ingredient for a recipe, where the recipe is indicative of a collection of multiple food items; and
executing, at the processor using the third set of instructions, the received set of entity preferences, the health label, and at least one of the ingredient for the recipe to determine a second health score, and upon the determined second score being within the predefined threshold, recommending, at the processor, the recipe for consumption to the entity.

14. The method of claim 1, wherein the health score is updated upon a change being determined in the received first set of input parameters and on receiving one or more instructions from the second entity.

15. The method of claim 1, wherein the one or more attributes of the entity corresponds to any or a combination of behavioral, emotional and physical characteristics of the entity.

16. A system for recommending a food item to an entity, said system comprising:
a processor of a remote computing device operatively coupled to a memory, the memory storing a first set of instructions and a second set of instructions executed by the processor to:
receive a first set of input parameters rᵢ associated with the entity, the first set of input parameters being indicative of one or more attributes of the entity and representative of one or more continuous variables;
receive a second set of input parameters from a second entity, the second set of input parameters being indicative of one or more health categories cⱼ, where the one or more health categories are associated with the first set of input parameters of the entity, the health categories being representative of one or more categorical variables;
analyze the received first set of input parameters and the received second set of input parameters to determine at least one of a health label for the entity;
assign a health score for at least one of the health label for the entity, the health score being assigned based on a food item to be recommended, the assignment being done on the execution of the second set of instructions; and
upon the assigned health score being within a predefined threshold, recommend the food item to the entity.

17. The system of claim 16, wherein the represented one or more continuous variables are real value numbers and are provided as input to the first set of instructions, and wherein the first set of input parameters representative of the one or more continuous variables are received using questionnaire data provided by the entity.

18. The system of claim 16, wherein the system further comprises:
extract a sequence of words from the food item to be recommend to the entity;
determine a plurality of similar words based on the extracted sequence of words using a third set of instructions, the determined plurality of similar words being indicative of the food item to be recommended to the entity, and wherein execute the third set of instructions to map the food item to be recommended to at least one of the health label; and
receive the health score for at least one of the health label, wherein upon the received health score being within the predefined threshold, recommend the food item to the entity.

19. The system of claim 18, wherein the determined plurality of similar words comprises at least one of a misspelling, synonym, abbreviation, metonym, synecdoche, metalepsis, kenning, or acronym associated with the extracted sequence of words.

20. The system of claim 18, wherein the system further comprises: determine a difference level between the extracted sequence of words and the determined plurality of similar words, and if the difference level between the extracted sequence of words and the at least one of determined plurality of similar words is below a threshold value considering the at least one of determined plurality of similar words as a closest match to the extracted sequence of words.
